(19) United States
(12) Patent Application Publication
(10) Pub. No.: US 2011/0245628 A1
(43) Pub. Date: Oct. 6, 2011

(54) Photoplethysmograph Filtering Using Empirical Mode Decomposition

(75) Inventors: Clark R. Baker, JR., Newman, CA (US); Edward M. McKenna, Boulder, CO (US); Daniel Peters, Longmont, CO (US); Youzhi Li, Longmont, CO (US)

(73) Assignee: Nellcor Puritan Bennett LLC, Boulder, CO (US)

(21) Appl. No.: 12/751,274

(22) Filed: Mar. 31, 2010

(51) Int. Cl. A61B 5/024; A61B 5/026; A61B 5/02; A61B 5/08; A61B 5/00

(52) U.S. Cl. 600/301; 600/504; 600/500; 600/508; 600/529; 600/300

(57) ABSTRACT

Present embodiments relate to systems, methods, and devices for decomposing a physiological signal of a patient using empirical mode decomposition (EMD). In one embodiment, the EMD algorithm may involve identifying a frequency component, referred to as an intrinsic mode function, in the physiological signal. The physiological signal may be decomposed into one or more intrinsic mode functions through multiple iterations of the EMD algorithm. Each subsequent mode function may have a different frequency component of the original physiological signal input into the EMD algorithm. In some embodiments, each mode function may be further analyzed and/or processed to determine various physiological data corresponding to blood flow in the patient.

### BACKGROUND

The present disclosure relates generally to non-invasive measurement of physiological parameters and, more particularly, using empirical mode decomposition to process physiological signals.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Pulse oximetry may be defined as a non-invasive technique that facilitates monitoring of a patient's blood flow characteristics. Specifically, these blood flow characteristic measurements may be acquired using a non-invasive sensor that passes light through a portion of a patient's tissue and photo-electrically senses the absorption and scattering of the light through the tissue. One or more physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered, which may be referred to as a plethysmograph waveform or a pulse oximetry signal, may then be used to estimate, for example, blood oxygen saturation of hemoglobin in a patient's arterial blood and/or the patient's heart rate.

However, typical algorithms used to calculate heart rate and/or blood oxygen saturation may not determine other physiological information which may be determinable from the plethysmograph waveform. In fact, as many physiological conditions may affect a patient's blood flow characteristics, the plethysmograph waveform may have signal characteristics which reflect various other physiological conditions. For example, in addition to oscillatory patterns corresponding to heart rate which may be found in the plethysmograph waveform, other oscillatory patterns which provide information on conditions such as respiratory rate, respiratory effort, heart arrhythmia, etc. may also be found in the plethysmograph waveform.

### SUMMARY

Certain aspects commensurate in scope with the originally disclosed embodiments are set forth below. It should be understood that these aspects are presented merely to provide the reader with a brief summary of certain forms the embodiments might take and that these aspects are not intended to limit the scope of the presently disclosed subject matter. Indeed, the embodiments may encompass a variety of aspects that may not be set forth below.

Present embodiments relate to systems, methods, and devices for decomposing a physiological signal of a patient using empirical mode decomposition (EMD). In one embodiment, the EMD algorithm may involve identifying a frequency component, referred to as an intrinsic mode function, in the physiological signal. The physiological signal may be decomposed into one or more intrinsic mode functions through multiple iterations of the EMD algorithm. Each subsequent mode function may have a different frequency component of the original physiological signal input into the EMD algorithm. Further, each mode function may be analyzed and/or processed to determine various physiological data corresponding to blood flow in the patient.

### BRIEF DESCRIPTION OF THE DRAWINGS

Advantages of the presently disclosed subject matter may become apparent upon reading the following detailed description and upon reference to the drawings in which:

FIG. 1 is a perspective view of a pulse oximeter system in accordance with an embodiment;

FIG. 2 is a block diagram of the pulse oximeter system of FIG. 1, in accordance with an embodiment;

FIG. 3 is a flow chart depicting a process for use by the system of FIG. 1 for decomposing a physiological signal using empirical mode decomposition, in accordance with an embodiment; and

FIG. 4 is a plot representing intrinsic mode functions obtained using the process of FIG. 3 on a physiological signal, in accordance with an embodiment.

### DETAILED DESCRIPTION

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to systems and methods of processing physiological signals corresponding to blood flow in a patient. Specifically, empirical mode decomposition (“EMD”) techniques may be applied to a physiological signal of the patient to decompose the signal into one or more components. The components decomposed from a signal may be referred to as “intrinsic mode functions,” which may each include a different frequency component of the original signal. Thus, each intrinsic mode function decomposed from a physiological signal may correspond to a physiological condition of the patient, including, for example, a pulse rate, respiratory rate, respiratory effort, sympathetic nervous activity, or any other repetitive variation affecting the patient's blood flow characteristics.

EMD may decompose a physiological signal, such that frequency components of the physiological signal (i.e., the intrinsic mode functions) may be analyzed within the time domain. In particular, the intrinsic mode functions may be analyzed with respect to time, such that the scale and frequency content of each mode function may vary in time. Furthermore, in accordance with the present techniques, the decomposition of the physiological signal is based only on the signal itself, and not on any predetermined frequencies or basis functions. Thus, the intrinsic mode functions obtained from the physiological signal represent the original frequency and scale content of the physiological signal with respect to time.

Using EMD may be particularly useful for physiological signals which may include frequency variations attributable to any number of physiological causes (e.g., pulse variations, respiratory variations, etc.). Such variations in a physiological signal may occur at specific times or in specific intervals, and analyzing the variations in the time domain may enable the determination of different causes for the variations in the physiological signal. Thus, EMD techniques may provide further physiological information not available under other methods of signal processing which transform time-domain signals out of the time domain and into the frequency domain or wavelet domain. For example, time information may not be preserved when using Fourier transforms, and certain physiological information may not be attainable by using only Fourier transforms to analyze a physiological signal.

A physiological signal may include a plethysmographic waveform, a pulse oximetry signal, or any other signal corresponding to blood flow in a patient. Physiological information determined from a physiological signal may include any repetitive variation in the patient which affects blood flow characteristics of the patient. For example, physiological information may include a pulse beat, respiratory rate, respiratory effort, sympathetic nervous activity, etc. Physiological information may also include less predictable variations corresponding to a patient's blood flow, which may be used to indicate heart arrhythmia or other heart irregularities.

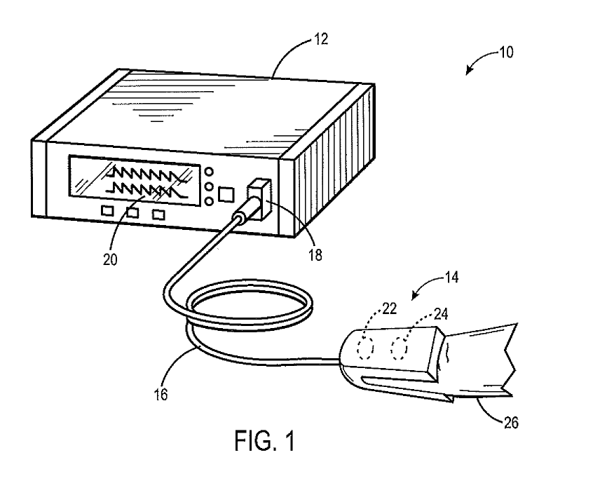

In one embodiment, a physiological signal such as a pulse oximetry signal may be obtained from a patient by using a pulse oximetry system. FIG. 1 illustrates a perspective view of a pulse oximetry system 10, which may include a patient monitor 12 and a pulse oximeter sensor 14. A sensor cable 16 may connect the sensor 14 to the patient monitor 12 via an electrical or optical connection 18. The sensor 14 may include an emitter 22 and a detector 24. The emitter 22 may emit a light beam into the pulsatile tissue of a patient 26. The emitted light may propagate through the pulsatile tissue, and the detector 24 may receive a resulting waveform from the pulsatile tissue of the patient 26 and guide the received waveform back to the patient monitor 12 via the sensor cable 16. The sensor 14 may be, for example, a reflectance-type sensor or a transmission-type sensor. Based on signals received from the sensor 14, the patient monitor 12 may determine certain physiological parameters that may appear on a display 20.

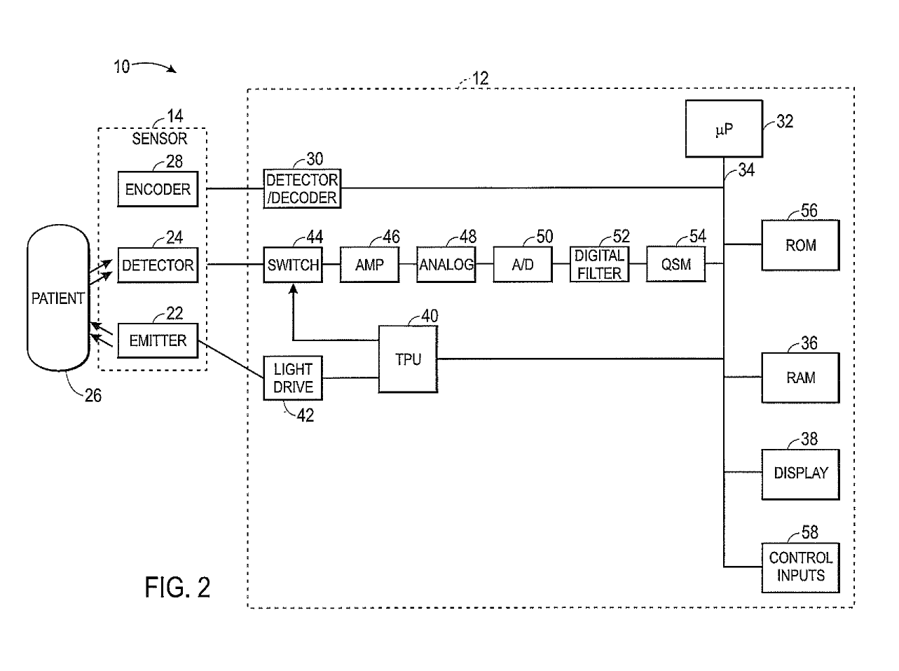

A simplified block diagram of a pulse oximeter system 10 is illustrated in FIG. 2, in accordance with an embodiment. Specifically, certain components of the sensor 14 and the monitor 12 are illustrated in FIG. 2. The sensor 14 may include an emitter 22, a detector 24, and an encoder 28. The emitter 22 may be capable of emitting at least two wavelengths of light, e.g., RED and infrared (IR) light, into the tissue of a patient 26, where the RED wavelength may be between about 600 nanometers (nm) and about 700 nm, and the IR wavelength may be between about 800 nm and about 1000 nm. The emitter 22 may include a single emitting device, for example, with two light emitting diodes (LEDs) or the emitter 22 may include a plurality of emitting devices with, for example, multiple LED's at various locations. Regardless of the number of emitting devices, the emitter 22 may be used to measure, for example, water fractions, hematocrit, or other physiologic parameters of the patient 26. As used herein, the term “light” may refer to one or more of ultrasound, radio, microwave, millimeter wave, infrared, visible, ultraviolet, gamma ray or X-ray electromagnetic radiation, and may also include modulated light, such as light modulated at sufficiently high frequencies (e.g., approximately 50 MHz to 3.0 GHz) to cause resolvable photon density waves to propagate through the patient's 26 tissue.

In one embodiment, the detector 24 may be capable of detecting light at various intensities and wavelengths. In operation, light enters the detector 24 after propagating through the tissue of the patient 26. The detector 24 may convert the light at a given intensity, which may be directly related to the absorbance and/or reflectance of light in the tissue of the patient 26, into an electrical signal. That is, when more light at a certain wavelength is absorbed or reflected, less light of that wavelength is typically received from the tissue by the detector 24. After converting the received light to an electrical signal, the detector 24 may send the signal to the monitor 12, where physiological characteristics may be calculated based at least in part on the absorption of light in the tissue of the patient 26. In some embodiments, physiological characteristics may also be calculated based in part on the scattering of light in the tissue of the patient 26. Furthermore, physiological characteristics may be determined based on one or more signal characteristics (oscillatory patterns) of the signal. The electrical signal converted by the detector 24 may also be referred to as a physiological signal, and may be in the form of a plethysmogram or any other representation corresponding to the light received from the patient 26 at the detector 24.

The sensor 14 may also include an encoder 28, which may contain information about the sensor 14, such as what type of sensor it is (e.g., whether the sensor is intended for placement on a forehead or digit) and the wavelengths of light emitted by the emitter 22. This information may allow the monitor 12 to select appropriate algorithms and/or calibration coefficients or to derive a filter for estimating the patient's physiological characteristics. The encoder 28 may, for instance, be a memory on which one or more of the following information may be stored for communication to the monitor 102. In some embodiments, the data or signal from the encoder 28 may be decoded by a detector/decoder 30 in the monitor 12.

Signals from the detector 24 and the encoder 28 may be transmitted to the monitor 12. The monitor 12 may include one or more processors 32 coupled to an internal bus 34. Also connected to the bus may be a RAM memory 36, ROM memory 56, and a display 38. A time processing unit (TPU) 40 may provide timing control signals to light drive circuitry 42, which controls when the emitter 22 is activated, and if multiple light sources are used, the multiplexed timing for the different light sources. TPU 40 may also control the gating-in of signals from detector 24 through a switching circuit 44. These signals are sampled at the proper time, depending at least in part upon which of multiple light sources is activated, if multiple light sources are used. The received signal from the detector 24 may be passed through an amplifier 46, an analog filter 48, and an analog-to-digital (A/D) converter 50, and/or a digital filter 52 for amplifying, filtering, digitizing, and/or processing the electrical signals from the sensor 14. After amplifying, filtering, digitizing, and/or processing, the digital data may then be stored in a queued serial module (QSM) 54, for later downloading to RAM 36 as QSM 54 fills up. In an embodiment, there may be multiple parallel paths for separate amplifiers, filters, and A/D converters for multiple light wavelengths or spectra received.

In some embodiments, based at least in part upon the physiological signal corresponding to the light provided by the detector 24, the processor 32 may use various algorithms to determine physiological information. The processor 32 may also access memory (e.g., RAM 36 or ROM 56) to access stored algorithms. In one or more embodiments, the processor 32 may apply algorithms such as empirical mode decomposition (EMD) algorithms, to extract frequency components from the physiological signal. The frequency components, also referred to as intrinsic mode functions or mode functions, may be analyzed to determine physiological information including, for example, pulse beat, respiration rate, respiratory effort, sympathetic nervous activity, or any other repetitive variation in heart rhythm.

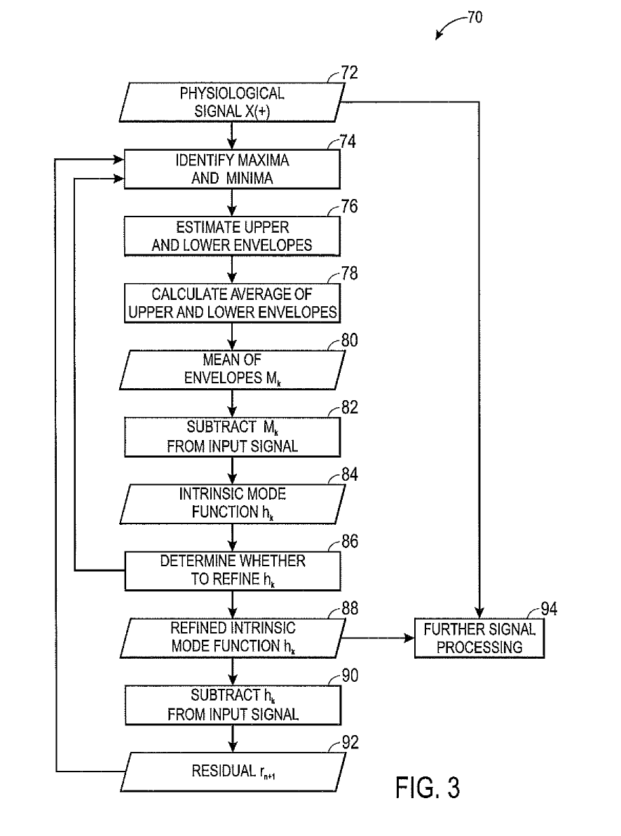

One embodiment of a process 70 for applying an empirical mode decomposition (EMD) algorithm to obtain intrinsic mode functions from a physiological signal is provided as a flow chart in FIG. 3. The process 70 may be applied to any physiological signal X(t) 72, including a pulse oximetry signal, a plethysmographic signal, or any other signal corresponding to blood flow in a patient. The physiological signal X(t) 72 may be a portion of the digitized signal generated by the detector 24 in the system 10 (as in FIG. 1). For example, the physiological signal X(t) 72 may span a window of time and may include certain number of samples. The window size of the physiological signal X(t) 72 may be selected by the processor 32, and may be based on the sampling interval of the detector 24 and/or a desired sample size of the physiological signal X(t) 72 to be decomposed. Furthermore, in some embodiments, the process 70 may be performed on overlapping time windows (e.g., a 20 second window that advances every second).

The process 70 may determine (block 74) the local maxima and minima of the input signal X(t) 72. The determination (block 74) of the local maxima and minima may be based on the type of intrinsic mode function to be extracted. For example, if an intrinsic mode function corresponding to a pulse rate is to be extracted from the physiological signal X(t) 72, the determination of the local maxima and minima may be designed to ignore artifacts substantially smaller than a typical or recent pulse amplitude. The physiological signal X(t) 72 may also include physiological signal characteristics which may not be useful in determining the pulse rate. For example, the dicrotic notch may not be a relevant signal characteristic for determining pulse rate. Thus, when the process 70 is extracting an intrinsic mode function corresponding to pulse rate, the determination (block 74) of the local maxima and minima of the physiological signal X(t) 72 may also be designed to ignore the dicrotic notch. Accounting for and ignoring artifacts and/or non-relevant physiological signal characteristics may be performed by the processor 32 using filters or any other suitable signal processing techniques. For embodiments involving multiple signals (e.g., multiple wavelength signals and/or signals from multiple detectors), timing information and clock cycles for the samples from each signal may be used to differentiate the multiple signals, such that the local maxima and minima of each of the multiple signals may be identified. As will be discussed, the process 70 may have more than one iteration using the output of the process 70 as a new input, and criteria for determining (block 74) the local maxima and minima may be modified at each subsequent iteration.

Furthermore, for embodiments using overlapping time windows, determining (block 74) the local maxima and minima of the physiological signal X(t) 72 may also involve using the maxima and minima information already determined in a previous time window. The previously determined maxima and minima may be compared with the new samples in the non-overlapping portion of the new window. Such a technique may save time in searching a previously analyzed window for local maxima and minima.

Once the local maxima and minima of the physiological signal X(t) 72 are identified, the process 70 may estimate (block 76) upper and lower envelopes based on the local maxima and minima. In one embodiment, upper and lower envelopes may be constructed by fitting cubic splines to the identified maxima and minima of the physiological signal X(t) 72. In estimating (block 76) the upper and lower envelopes, the process 70 may account for local maxima and minima not occurring at the beginning and/or end of the window of physiological signal X(t) 72. For example, estimating (block 76) the upper and lower envelopes may duplicate the nearest identified maxima and minima at the beginning and/or end of the data window. In estimating (block 76) the upper and lower envelopes, the process may also compensate for changes in the physiological signal X(t) 72 which may be due to non-physiological causes, such as adjustments of the internal gain of the pulse oximetry system 10, adjustments in the source intensity (e.g., from the emitter 22 and/or light drive 42 of the system 10), and/or periods of interruption in the physiological signal, such as during sensor 14 adjustments or during periods when the sensor 14 is disconnected. Furthermore, in embodiments involving multiple signals (e.g., multiple wavelength signals and/or signals from multiple detectors), timing information and clock cycles for the samples from each signal may be used, such that cubic splines may be fitted for the appropriate data values of each respective signal.

Once the upper and lower envelopes have been estimated (block 76), the process 70 may then calculate (block 78) the mean mₖ 80 of the upper and lower envelopes. By subtracting (block 82) the mean mₖ 80 of the upper and lower envelopes from the original physiological signal X(t) 72, the process 70 produces an intrinsic mode function hₖ 84. This relationship is represented in equation (1), below:

X(t)−mₖ=hₖ equation (1)

By definition, the intrinsic mode function hₖ 84 may have the same number of extrema (i.e., maxima and minima) as the physiological signal X(t) 72, and may represent an oscillatory mode of the physiological signal X(t) 72. As discussed, the physiological signal X(t) 72 may be a representation of blood flow in a patient 26, which may include one or more oscillatory patterns (e.g., oscillatory concentrations of blood cells, oscillatory ratios of oxygenated to deoxygenated hemoglobin, etc.) resulting from certain physiological conditions of the patient 26. The empirical mode decomposition process 70 may identify such repeating signal characteristics by decomposing the physiological signal X(t) 72 into intrinsic mode functions hₖ 84. As a intrinsic mode function hₖ 84 is decomposed from the original physiological signal X(t) 72 without leaving the time domain, the original scale of the intrinsic mode function hₖ 84 may be preserved in time. Thus, in some embodiments, the intrinsic mode function hₖ 84 may be further analyzed and/or processed with respect to time. Retaining time information may be valuable when analyzing physiological signals, as the timing of physiological causes may be important in identifying certain conditions of the patient 26.

In some embodiments, the process 70 may include iterative refinement of each intrinsic mode function hₖ 84, which may involve repeating the steps 74, 76, 78, and 82 until the process determines (block 86) that the intrinsic mode function hₖ 84 is refined. If the intrinsic mode function hₖ 84 is determined to be not sufficiently refined, the intrinsic mode function hₖ 84 may be subtracted from the input signal, and steps 74, 76, 78, and 82 may be performed on the residual of this subtraction. Iterations of this portion of the process 70 may be performed until the intrinsic mode function hₖ 84 is sufficiently refined.

Determining (block 86) whether the intrinsic mode function hₖ 84 is sufficiently refined may involve comparing a statistical measure of an intrinsic mode function hₖ 84 to a predetermined threshold and/or to a statistical measure of an intrinsic mode function hₖ 84 from a previous iteration (e.g., comparing statistical measures of h₂ and h₃). Such statistical measures may include calculating the kurtosis of an intrinsic mode function hₖ 84, which should asymptotically decrease as lower-frequency modes are decomposed from the signal. For example, a higher kurtosis may indicate that more of the variance of an intrinsic mode function hₖ 84 is a result of relatively infrequent and extreme deviations (which may be more indicative of noise or other non-physiological conditions), as opposed to a more frequent and less extreme deviation (which may be more indicative of a physiological characteristic). Some embodiments may also involve statistical measures such as quantifying the variability in the amplitude, maxima, or minima of the input signal. Furthermore, some embodiments may include determining the number of minima, maxima, zero crossings, or any other indication of the number of cycles expressed by an intrinsic mode function hₖ 84.

In some embodiments, the skewness of the derivative of an intrinsic mode function hₖ 84 may also be used to determine whether substantially all of the oscillatory content of the physiological signal X(t) 72 has been decomposed. For example, the skewness of the derivative of a mode should decrease as the physiological signal X(t) 72 waveform is refined and as mode estimates are decomposed from the signal X(t) 72. Once the skewness of a mode estimate does not decrease when compared to a previous mode estimate, then the intrinsic mode function hₖ 84 may be refined, as indicated by the refined intrinsic mode function hₖ 88. It should be noted that in some iterations of the process 70, the intrinsic mode function hₖ 84 may be determined (block 86) to be sufficiently refined. Thus, in some iterations of the process 70, the intrinsic mode function hₖ 84 may be the same as the refined intrinsic mode function hₖ 88, and the refined intrinsic mode function hₖ 88 may simply be referred to henceforth as the intrinsic mode function hₖ 88.

The process 70 may involve finding more than one intrinsic mode function hₖ 88, as a patient's 26 blood flow may be affected by more than one system (e.g., circulatory system and respiratory system), and the physiological signal X(t) 72 may include more than one oscillatory mode. For example, the first intrinsic mode function hₖ 88 found from the physiological signal X(t) 72 may be referred to as an intrinsic mode function h₀. To find a subsequent intrinsic mode function h₁, the intrinsic mode function h₀ may be subtracted (block 90) from the original physiological signal X(t) 72, resulting in the residual rₙ₊₁ 92, as represented in the equation below:

X(t)−hₖ=rₙ₊₁ equation (2)

The residual rₙ₊₁ 92 may then be used as the input signal for each subsequent iteration (where the k of hₖ represents the iteration number) of the process 70, and the maxima and minima of the residual rₙ₊₁ 92 may be identified (block 74). As discussed, the maxima and minima identification for each subsequent residual rₙ₊₁ 92 may be modified according to typical characteristics of the physiological signal X(t) 72, the number of iterations k, the number of intrinsic mode functions hₖ 88 already calculated, and/or the type of intrinsic mode function hₖ 88 to be extracted from the physiological signal X(t) 72.

As the intrinsic mode function h₀ has already been subtracted (block 90) from the physiological signal X(t) 72 to produce the residual rₙ₊₁ 92, the remaining features in the residual may be less extreme than the features of the physiological signal X(t) 72. For example, the maxima identified in the residual rₙ₊₁ 92 may be smaller than the previously identified maxima of the physiological signal X(t) 72, and the minima identified in the residual rₙ₊₁ 92 may be larger than the previously identified minima of the physiological signal X(t) 72. Thus, subsequent iteration of the process 70 (iteration k=1) on the residual rₙ₊₁ 92 may produce an intrinsic mode function h₁ having a lower order frequency compared to the first intrinsic mode function h₀. Each subsequent intrinsic mode function hₖ 88 may represent a progressively lower order frequency component of the physiological signal X(t) 72, and the sum of all identified intrinsic mode functions hₖ 88 of a physiological signal X(t) 72 may be approximately equal to the total oscillatory content of the physiological signal X(t) 72. In other words, each subsequent iteration of the EMD algorithm may produce an intrinsic mode function hₖ 88 having the next most distinguishing features of the physiological signal X(t) 72, and when the physiological signal X(t) 72 has been refined (i.e., decomposed), all of the distinguishing features may be extracted in the form of intrinsic mode functions hₖ 88.

In some embodiments, the process 70 may continue until substantially all of the oscillatory content of the physiological signal X(t) 72 is decomposed into intrinsic mode functions hₖ 88. For example, methods of determining whether substantially all of the oscillatory content in the physiological signal X(t) 72 waveform has been sufficiently decomposed may involve analyzing each residual rₙ₊₁ 92 and determining whether the residual rₙ₊₁ 92 is smaller than a predetermined value, or whether it is a monotonic function. If the residual rₙ₊₁ 92 is smaller than a predetermined value and/or if the residual rₙ₊₁ 92 is a monotonic function, then the process 70 may have identified substantially all the intrinsic mode functions hₖ 84 of the physiological signal X(t) 72.

In one embodiment, the number of iterations in the process 70 (and the number of intrinsic mode functions hₖ 88 extracted) may also be based on the type of physiological information to be determined from the intrinsic mode functions hₖ 88. For example, the process 70 may have substantially refined the physiological signal X(t) 72 once all relevant intrinsic mode functions hₖ 88 have been extracted. In some embodiments, the process 70 may still continue to provide further estimations of intrinsic mode functions hₖ 88 to account for changes in the signal detected by the detector 24 (e.g., signal interruptions, system 10 changes, etc.), and/or to provide more accurate estimates of the intrinsic mode functions hₖ 88.

Performing the process 70 on a physiological signal may decompose the signal into multiple intrinsic mode functions hₖ 88. In some embodiments, the multiple intrinsic mode functions hₖ 88 may each be further processed (block 94) to determine various physiological information, if any, indicated by each extracted mode function. In accordance with the present techniques, any suitable signal processing techniques may be combined with the EMD algorithm to further enhance the utilization of the intrinsic mode functions and/or aid in the determination of physiological parameters and indications. Signal processing may be performed on any extracted mode function, and may include comparisons of any mode function with a pre-decomposed physiological signal.

Signal processing may be performed by any suitable processor (e.g., microprocessor 32) in the system 10, and may include other elements in the system 10 (FIG. 2). For example, signal processing techniques may include calibration of the system 10, power-saving techniques, multiplexing, amplification, and/or digitization of signals. Specific conditions of the system 10 and/or the patient 26 from which a physiological signal is being measured may also be used to process signals in some embodiments. For example, calculations may be made based on a type of sensor 14 used, a measurement site of the sensor 14 on the patient 26, and/or a physiological condition of a patient 26. Determinations may also be made as to whether the sensor 14 is applied to an appropriate tissue site on the patient 26. In addition, certain physiologic assumptions may also be used, including limits on typical and/or possible ranges of a physiological parameter or a rate of change of a physiological parameter.

In some embodiments, signal processing techniques (block 94) may also involve linear and/or non-linear filters which may be adjustable or adaptable based on one or more metrics, trends, patterns, and/or distributions of the inputs or outputs of the filters. Such filters may include, for example, Kalman filters, adaptive comb filters, adaptive noise cancellers, joint process filters, and lattice filters. Furthermore, a physiological signal and/or a mode function of the physiological signal may be normalized, resealed, and/or transformed in the frequency and/or wavelet domains. Various techniques may also be used for computing ratios or other combinations of the components (e.g. from multiple wavelengths or detectors) of the physiological signal or intrinsic mode functions extracted from the physiological signal. For example, such techniques may include linear regression, linear combination, multivariate analysis, principal component analysis (PCA), other suitable matrix techniques, or independent component analysis (ICA). Furthermore, signal processing techniques may include use of neural nets, fuzzy logic, genetic-based algorithms, or any other learning-based algorithms. Analysis of parallel or alternative estimates or algorithms, such as a Hidden Markov Model, may also be used.

Signal processing techniques (block 94) may include the combination of a physiological signal with additional sensors, including motion, pressure, temperature, or ultrasound sensors. The additional sensors may provide data to be used with the physiological signal which may aid in distinguishing physiological signals from artifacts or other non-physiological components. Furthermore, the empirical mode decomposition algorithm used herein may be used along with Hilbert Spectral Analysis in the Hilbert-Huang Transform, but is not limited to this combination of techniques.

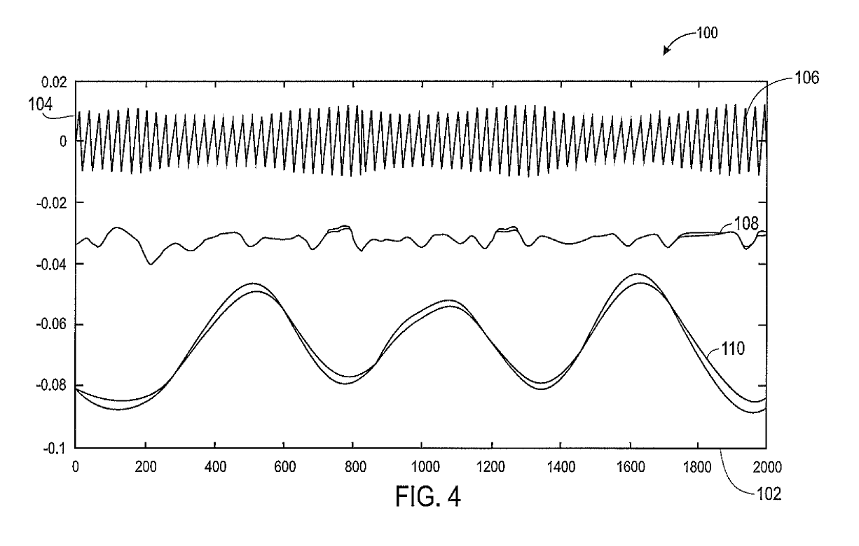

Turning now to FIG. 4, the graph 100 provides examples of three intrinsic mode functions which may be decomposed from a physiological signal. The graph 100 depicts the amplitude 104 and time course of each mode function 106, 108, and 110 over 2000 samples 102. For example, the sampling interval may be approximately 17.5 ms, and a 2000 sample window may be approximately 35 seconds long. A first mode function 106 may typically represent the pulse rate, which may be approximately 100 beats per minute. The first mode function 106 may have the highest degree of oscillatory content in the physiological signal from which it has been decomposed.

The second mode function 108 illustrated in the graph 100 may represent another repetitive variation in heart rhythm. For example, the second mode function 108 may contain indications of arrhythmia, and could contain a waveform at approximately half the frequency of a pulse rate. Analyzing the waveform of the extracted mode function may also enable a health practitioner to determine clinical conditions, such as, for example, bi-Gemini, which may appear as alternating large and small pulses. Furthermore, in the absence of waveform characteristics indicative of heart rhythm, the second mode function 108 may also indicate the patient's 26 respiration, as respiratory related changes in intra-thoracic pressure may also impact the rate at which venous blood flows from peripheral to central venous circulation. The third mode function 110 may contain a waveform indicative of the patient's 26 respiration, if respiration is not already contained in a previous mode function. Alternatively, the third mode function 110 could reflect sympathetic nervous activity, such as Mayer waves.

The physiological information determined based on each mode function may not always follow a particular order, and may follow a different order from the examples given above. Further, not all extracted mode functions may provide physiological information. For example, in some situations, the decomposition of any of the mode functions may sometimes be affected by artifacts which may be mistaken for maxima and minima. Such motion artifacts may appear in any mode, depending on their frequency content and the relationship of their frequency to that of physiological signals. For example, high-frequency artifacts may appear in a first mode function. Such artifacts may be identified and/or reduced by using signal processing techniques as discussed with respect to FIG. 3.

Furthermore, physiological parameters and indications may not be limited to the examples provided, and may include any physiological condition capable of affecting a patient's blood flow characteristics. For example, a physiological parameter or indication which may be determined using the present techniques may include arterial or venous oxygen saturation, pulse rate, continuous non-invasive blood pressure, pulse transit time, respiratory rate or effort, pulse amplitude, tissue perfusion, hypoxia, hyperoxia, bradycardia, tachycardia, arrhythmia, central or obstructive apnea, hypopnea, Cheyne-Stokes syndrome, hypovolemia, or sympathetic nervous activity (e.g., Mayer waves).

While the embodiments set forth in the present disclosure may be susceptible to various modifications and alternative forms, specific embodiments have been shown by way of example in the drawings and have been described in detail herein. However, it should be understood that the disclosure is not intended to be limited to the particular forms disclosed. The disclosure is to cover all modifications, equivalents, and alternatives falling within the spirit and scope of the disclosure as defined by the following appended claims.

What is claimed is:

1. A method comprising:
applying an empirical mode decomposition (EMD) algorithm on a physiological signal to produce one or more intrinsic mode functions, wherein the physiological signal corresponds to blood flow in a patient; and
determining one or more physiological parameters based on the one or more intrinsic mode functions.

2. The method of claim 1, wherein applying the EMD algorithm comprises:
identifying a maxima and a minima of the physiological signal;
calculating an upper envelope and a lower envelope based on the maxima and the minima; and
subtracting a mean of the upper envelope and the lower envelope from the physiological signal to produce a first mode of the one or more intrinsic mode functions.

3. The method of claim 2, wherein determining one or more physiological parameters comprises determining a pulse rate of the patient based on the first mode.

4. The method of claim 2, comprising:
subtracting the first mode from the physiological signal to produce a residual;
identifying a maxima and a minima of the residual;
calculating an upper envelope and a lower envelope of the residual based on the maxima and the minima of the residual; and
subtracting a mean of the upper envelope and the lower envelope of the residual to produce a second mode of the one or more intrinsic functions.

5. The method of claim 4, wherein determining one or more physiological parameters comprises determining a heart arrhythmia or a respiratory rate of the patient based on the second mode.

6. The method of claim 1, comprising processing the one or more intrinsic mode functions by performing one or more of multiplexing, amplifying, digitizing, filtering, normalizing, resealing, and transforming the one or more intrinsic mode functions.

7. The method of claim 1, wherein determining the one or more physiological parameters comprises computing a ratio of pulse amplitudes by using one or more of linear regression techniques, linear combination techniques, multivariate analysis, principal component analysis, and independent component analysis.

8. The method of claim 1, wherein the one or more physiological parameters comprises one or more of arterial or venous oxygen saturation, pulse rate, continuous non-invasive blood pressure, pulse transit time, respiratory rate or effort, pulse amplitude, tissue perfusion, hypoxia, hyperoxia, bradycardia, tachycardia, arrhythmia, central or obstructive apnea, hypopnea, Cheyne-Stokes syndrome, hypovolemia, and sympathetic nervous activity.

9. A method of determining physiological information of a patient comprising:
identifying extrema in an input signal, wherein the input signal comprises a portion of a physiological signal of the patient;
calculating input signal envelopes based on the extrema of the input signal;
subtracting a mean of the input signal envelopes from the input signal to produce a first mode function;
subtracting the first mode function from the input signal to produce a residual signal;
identifying extrema in the residual signal;
calculating residual signal envelopes based on the extrema of the residual signal;
subtracting a mean of the residual signal envelopes from the residual signal to produce a second mode function; and
determining physiological information of the patient based on one or more of the first mode function and the second mode function.

10. The method of claim 9, wherein the input signal comprises a time window of samples from the physiological signal.

11. The method of claim 10, wherein the method is performed on a subsequent time window of samples from the physiological signal, wherein the subsequent time window overlaps with the time window.

12. The method of claim 9, wherein identifying the extrema in the input signal comprises ignoring samples in the input signal corresponding to a dicrotic notch of the patient.

13. The method of claim 9, wherein identifying the extrema in the input signal and identifying the extrema in the residual signal comprises ignoring samples not relevant to the physiological information to be determined by the first mode function and the second mode function.

14. The method of claim 9, wherein a variance of the second mode function is compared with a variance of the first mode function, and wherein an additional iteration is performed to produce a refined second mode function if the variance of the second mode function is less than the variance of the first mode function, wherein the additional iteration comprises:
subtracting the second mode function from the residual signal to produce a second residual;
identifying extrema in the second residual;
calculating second residual envelopes based on the extrema of the second residual; and
subtracting a mean of the second residual envelopes from the second residual to produce a refined second mode function.

15. The method of claim 9, comprising:
determining whether to refine the first mode function based on a comparison of statistical measures of the first mode function with threshold statistical measures; and
performing one or more iterations to produce a refined first mode function, wherein the one or more iterations each comprise:
subtracting the first mode function from the input signal to produce an unrefined residual signal;
identifying extrema in the unrefined residual signal;
calculating unrefined residual signal envelopes based on the extrema of the unrefined residual signal; and
subtracting a mean of the unrefined residual signal envelopes from the unrefined residual signal to produce a refined first mode function.

16. The method of claim 15, wherein the statistical measures comprise one or more of a variance, a kurtoses, a skewness, a number of minima, a number of maxima, a number of zero crossings, and a number of cycles of the first mode function.

17. The method of claim 9, comprising calculating subsequent mode functions until a number of cycles of a mode function is below a threshold.

18. The method of claim 9, wherein determining physiological information of the patient comprises comparing one or more of the first mode function, the second mode function, and the input signal.

19. A system for determining physiological information of a patient, the system comprising:
a sensor configured to detect a physiological signal from the patient;
a patient monitor coupled to the sensor, wherein the patient monitor comprises:
a processor configured to apply an empirical mode decomposition (EMD) algorithm on the physiological signal to produce one or more intrinsic mode functions; and
a processor configured to process the one or more intrinsic mode functions to determine one or more physiological parameters.

20. The system of claim 19, wherein the processor is configured to apply the EMD algorithm iteratively on the physiological signal to produce subsequent intrinsic mode functions.

21. The system of claim 19, wherein the processor is configured to iteratively apply the EMD algorithm on the physiological signal until the one or more intrinsic mode functions represent substantially all physiological oscillations in the physiological signal.

22. The system of claim 19, wherein the processor is configured to:
calculate statistical measures of the one or more intrinsic mode functions;
compare the calculated statistical measures with threshold statistical measures;
determine whether each of the one or more intrinsic mode functions is sufficiently refined based on the comparison; and
iteratively apply portions of the EMD algorithm on each of the one or more intrinsic mode functions until each of the one or more intrinsic mode functions is determined to be sufficiently refined.

23. The system of claim 19, wherein the processor is configured to compare a first mode of the one or more intrinsic mode functions with a second mode of the one or more intrinsic mode functions, and wherein the processor is configured to produce a third mode of the one or more intrinsic mode functions based on a comparison of the first mode and the second mode.
